(19) United States
(12) Patent Application Publication
(10) Pub. No.: US 2019/0353592 A1
(43) Pub. Date: Nov. 21, 2019

(54) APPARATUS AND METHOD USING SURFACE PLASMON RESONANCE

(71) Applicant: ELECTRONICS AND TELECOMMUNICATIONS RESEARCH INSTITUTE, Daejeon (KR)

(72) Inventors: Won Kyoung LEE, Daejeon (KR); Bong Kyu KIM, Daejeon (KR); Hong-Seok SEO, Daejeon (KR)

(21) Appl. No.: 16/216,391

(22) Filed: Dec. 11, 2018

(30) Foreign Application Priority Data
May 15, 2018 (KR) 10-2018-0055625

(51) Int. Cl. G01N 21/552; G01N 33/543

(52) U.S. Cl. CPC G01N 21/554; G01N 2201/0826; G01N 33/54373

(57) ABSTRACT

Disclosed herein are a sensor and a system for measuring an analyte using surface plasmon resonance. The sensor may include: an optical fiber including a core layer, and a plasmon resonance layer which is formed to surround an outer surface of the core layer and on which the analyte is disposed; an acoustic wave perturbation generator connected to one side of the optical fiber, and generating acoustic wave perturbation to a mode which enters into the core layer to allow the mode to exit the plasmon resonance layer; and a detector for detecting the mode passing through the inside of the core layer.

### CROSS-REFERENCE TO RELATED APPLICATION

This application claims priority to and the benefit of Korean Patent Application No. 10-2018-0055625 filed in the Korean Intellectual Property Office on May 15, 2018, the entire contents of which are incorporated herein by reference.

### BACKGROUND OF THE INVENTION

### (a) Field of the Invention

The present invention relates to a surface plasmon resonance sensor based on an optical fiber using acoustic wave perturbation.

### (b) Description of the Related Art

As the biosensor market such as point-of-care (POC) and virus examination expands with the development of nanotechnology, biosensor technology based on surface plasmon resonance (SPR) has attracted attention in various applications. In addition, the biosensor technology based on SPR is developing into a lab-on-a-chip form. Particularly, localized surface plasmon resonance (LSPR) technology among the biosensor technologies has been actively studied in accordance with a demand for miniaturization and integration of sensors.

Cost-effectiveness, portability, easy-to-use, high sensitivity, and real-time monitoring functions are required to enable SPR biosensors to enhance competitiveness in the biosensor market.

Traditional SPR biosensors having high sensitivity have a problem in that they are bulky and expensive, whereas traditional SPR biosensors having a small volume have a problem in that they have low sensitivity and resolution.

### SUMMARY OF THE INVENTION

According to an embodiment of the present disclosure there is provided a sensor for measuring an analyte using surface plasmon resonance, the sensor including: an optical fiber including a core layer, and a plasmon resonance layer which is formed to surround an outer surface of the core layer and on which the analyte is disposed; an acoustic wave perturbation generator connected to one side of the optical fiber, and generating acoustic wave perturbation to a mode which enters into the core layer to allow the mode to exit the plasmon resonance layer; and a detector for detecting the mode passing through inside of the core layer.

The plasmon resonance layer may be formed of a metallic colloid.

The plasmon resonance layer may include a cladding layer formed to surround the outer surface of the core layer, and a coating layer formed to surround the outer surface of the cladding layer.

The coating layer may be formed of a metal film.

The coating layer may be formed of a plasmonic nanostructure.

The acoustic wave perturbation generator may include a metal block having a first through hole; an oscillator having a second through hole passing through one side and the other side of the oscillator, wherein the second through hole of the one side is joined to the first through hole, and wherein the second through hole of the other side is joined to the core layer of one side of the optical fiber; and a signal generator for transmitting a signal to the oscillator to generate acoustic wave perturbation to a mode which passes through the first through hole and the second through hole and which enters into the core layer.

The sensor may further include an acoustic damper connected to the other side of the optical fiber to eliminate the acoustic wave perturbation.

The plasmon resonance layer may include a cladding layer formed to surround the outer surface of the core layer; and a plurality of coating layers spaced apart from each other by a predetermined distance, and formed to surround the outer surface of the cladding layer.

The plurality of coating layers may be formed of a metal film.

The plurality of coating layers may be formed of a plasmonic nanostructure.

The acoustic wave perturbation generator may include a metal block having a first through hole; an oscillator having a second through hole passing through one side and the other side of the oscillator, wherein the second through hole of the one side is joined to the first through hole, and wherein the second through hole of the other side is joined to the core layer of one side of the optical fiber; and a signal generator for transmitting a signal to the oscillator to generate acoustic wave perturbation to a mode which passes through the first through hole and the second through hole and which enters into the core layer.

The sensor may further include an acoustic damper connected to the other side of the optical fiber to eliminate the acoustic wave perturbation.

According to an embodiment of the present disclosure there is provided A system for sensing a plurality of analytes using a plurality of surface plasmon resonance sensors, the system including: a plurality of surface plasmon resonance sensors each including an optical fiber including a core layer, and a plasmon resonance layer which is formed to surround an outer surface of the core layer and on which the analytes are disposed, and an acoustic wave perturbation generator connected to one side of the optical fiber and generating acoustic wave perturbation to a mode which enters into the core layer to allow the mode to exit the plasmon resonance layer; a demultiplexer for distributing light from a light source to the plurality of surface plasmon resonance sensors; and a controller for operating the demultiplexer so that the light is distributed to each of the plurality of surface plasmon resonance sensors, and for operating the acoustic wave perturbation generator so that the acoustic wave perturbation occurs in the light distributed to each of the plurality of surface plasmon resonance sensors from the demultiplexer.

The demultiplexer and each of the plurality of surface plasmon resonance sensors may be connected by optical fibers having different lengths.

The plasmon resonance layer may be formed of a metal colloid.

The plasmon resonance layer may include a cladding layer formed to surround the outer surface of the core layer; and a coating layer formed to surround the outer surface of the cladding layer.

The coating layer may be formed of a metal film.

The coating layer may be formed of a plasmonic nanostructure.

### BRIEF DESCRIPTION OF THE DRAWINGS

FIG. 1 and FIG. 2 are schematic diagrams illustrating surface plasmon resonance principle.

FIG. 3 to FIG. 6 are schematic diagrams illustrating a surface plasmon resonance sensor using a prism.

FIG. 7 to FIG. 11 are schematic diagrams illustrating a surface plasmon resonance sensor using an optical fiber.

FIG. 12 is a block diagram illustrating a surface plasmon resonance sensor using acoustic wave perturbation according to an exemplary embodiment.

FIG. 13 is a schematic diagram illustrating a surface plasmon resonance sensor using acoustic wave perturbation according to an exemplary embodiment.

FIG. 14 and FIG. 15 are schematic diagrams illustrating changes in permittivity and angle of surface plasmon resonance according to a metal type.

FIG. 16 and FIG. 17 are schematic diagrams illustrating changes in a propagation constant of incident light and a phase matching condition by acoustic wave perturbation of a surface plasmon resonance sensor according to an exemplary embodiment.

FIG. 18 is a schematic diagram illustrating a multi-channel surface plasmon resonance sensor using acoustic wave perturbation according to an exemplary embodiment.

FIG. 19 is a schematic diagram illustrating a spectrum of a multi-channel surface plasmon resonance sensor using acoustic wave perturbation according to an exemplary embodiment.

FIG. 20 is a schematic diagram illustrating a multi-channel surface plasmon resonance sensing system using acoustic wave perturbation and time delay according to an exemplary embodiment.

FIG. 21 and FIG. 22 are schematic diagrams illustrating a spectrum of a multi-channel surface plasmon resonance sensing system using acoustic wave perturbation and time delay according to an exemplary embodiment.

### DETAILED DESCRIPTION OF THE EMBODIMENTS

Hereinafter, exemplary embodiments of the present disclosure will be described in detail with reference to the accompanying drawings so that those skilled in the art may easily practice the present disclosure. However, the present disclosure may be modified in various different ways, and is not limited to embodiments described herein. In the accompanying drawings, portions unrelated to the description will be omitted in order to obviously describe the present disclosure, and similar reference numerals will be used to describe similar portions throughout the present specification.

Throughout the present specification and the claims, unless explicitly described to the contrary, the word “comprise” and variations such as “comprises” or “comprising” will be understood to imply the inclusion of stated elements but not the exclusion of any other elements.

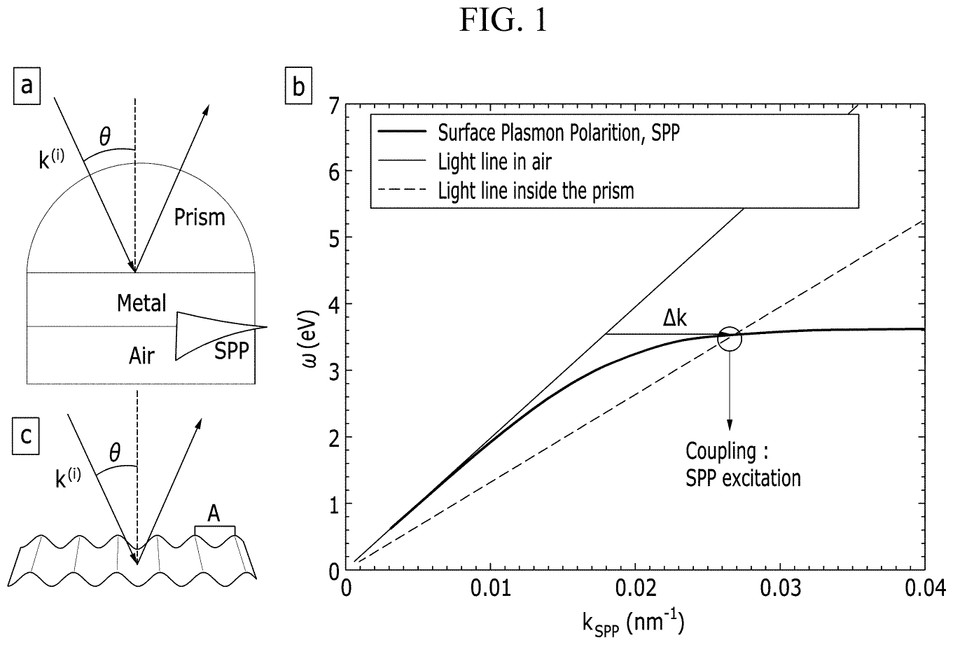

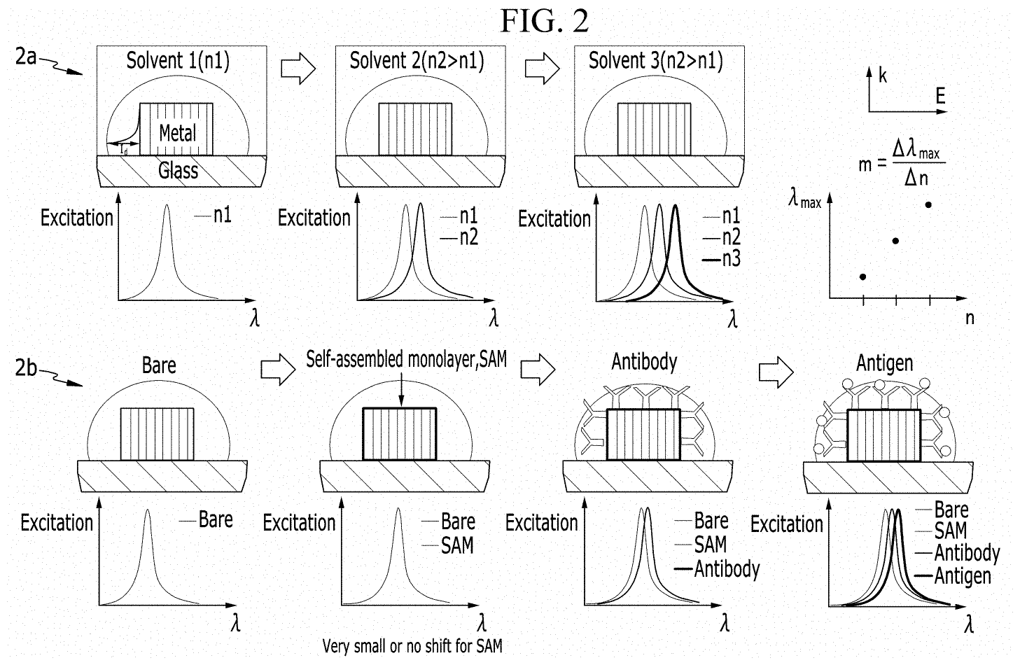

FIG. 1 and FIG. 2 are schematic diagrams illustrating surface plasmon resonance principle.

Referring to FIG. 1, the surface plasmon resonance (SPR) is a phenomenon in which a part of energy of incident light is transmitted to a surface plasmon when a resonance occurs due to matching of a propagation constant of the incident light and a dielectric constant of oscillation of free electrons of a metal surface.

A condition of the propagation constant for the resonance is shown in Equation 1, and a condition of coupling for the resonance is shown in Equation 2.

$$\begin{matrix}{k}_{sp} = {{k}_{o} ( \frac{{ɛ}_{m} {ɛ}_{s}}{{ɛ}_{m} + {ɛ}_{s}} )}^{1 / 2} , {ɛ}_{m} = Re ( {ɛ}_{m} ) + Im ( {ɛ}_{m} ) & [ Equation 1 ] \\ k = {k}_{o} \sqrt{{ɛ}_{p}} sin ( θ ) , k = {k}_{sp} & [ Equation 2 ]\end{matrix}$$

Herein, kₛₚ is a propagation constant of surface plasmon, k₀ is a propagation constant in the atmosphere, k is a component of the propagation constant of the incident light in a same direction as a surface, εₘ is permittivity of a metal, εₛ is a dielectric constant of a dielectric such as a prism, and θ is an incident angle.

Referring to FIG. 2, the surface plasmon resonance sensor may monitor binding events between biomolecules using changes in incident angle or wavelength.

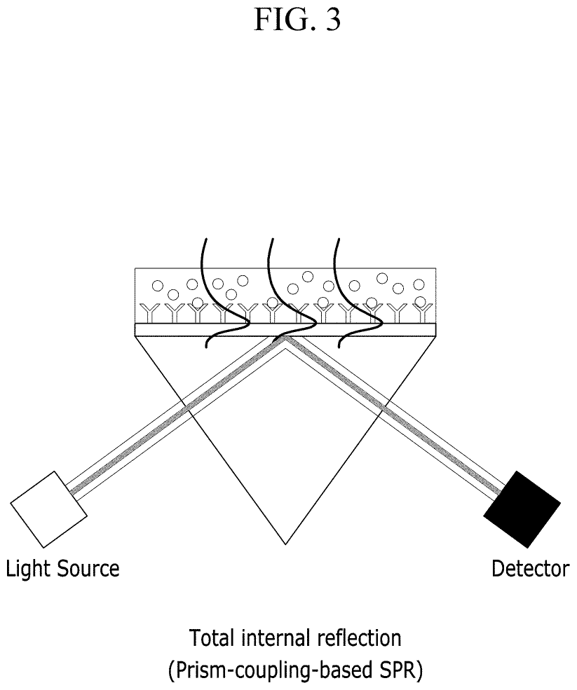

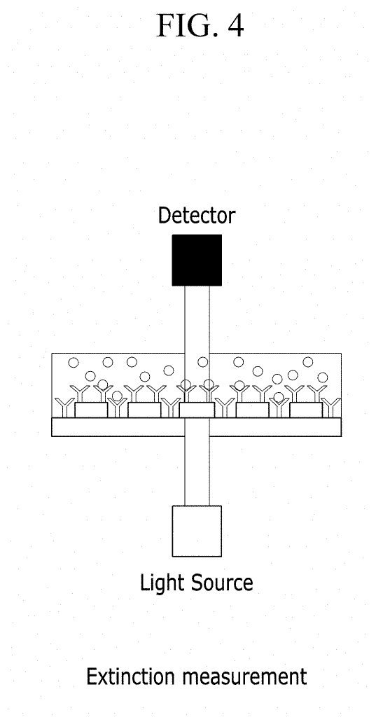

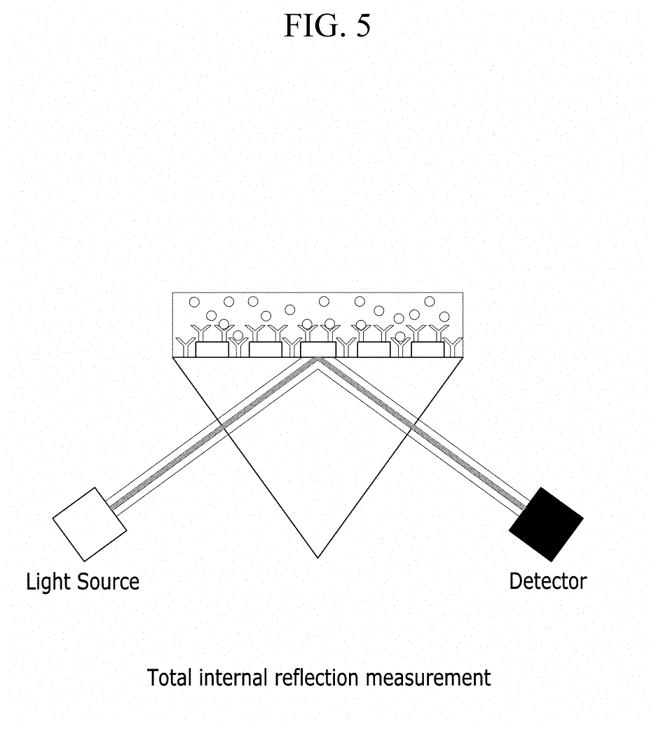

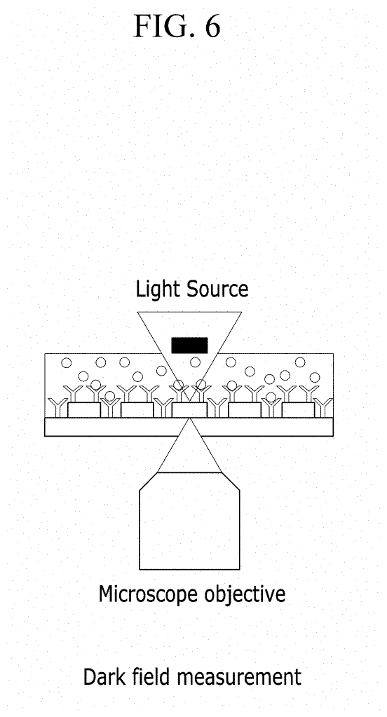

FIG. 3 to FIG. 6 are schematic diagrams illustrating a conventional surface plasmon resonance sensor.

Referring to FIG. 3 to FIG. 6, the conventional surface plasmon resonance sensor has a structure for measuring a reflection or transmission signal of a signal incident on the prism in order to confirm a resonance angle and a resonance wavelength. The conventional surface plasmon resonance sensor is required to have materials and structures capable of optimizing characteristics of a nanostructure for largely sensitive change of a refractive index by chemical binding of incident light and biomolecules.

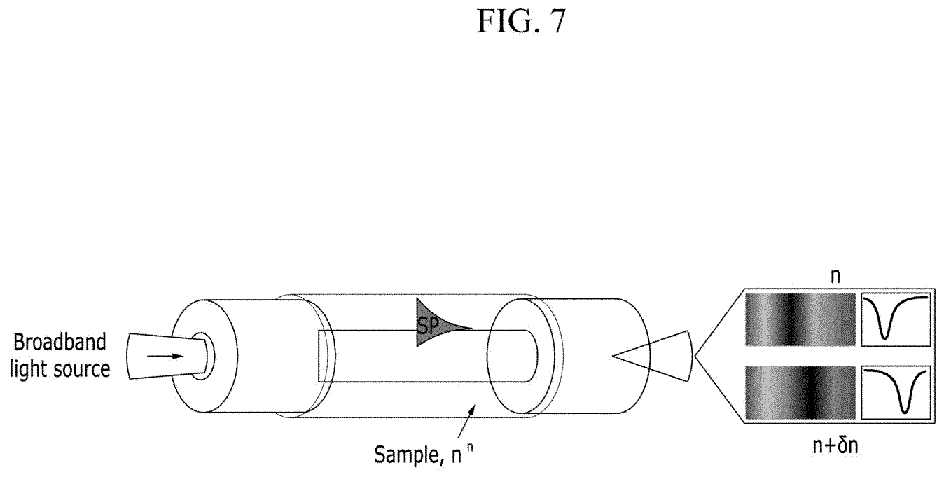

FIG. 7 to FIG. 11 are schematic diagrams illustrating a surface plasmon resonance sensor using an optical fiber.

Referring to FIG. 7, the surface plasmon resonance sensor using an optical fiber has a structure that replaces a prism of the conventional surface plasmon resonance sensor. Coupling conditions of the surface plasmon resonance sensor using an optical fiber can be changed by a size of an optical fiber core, a refractive index size, and an optical fiber structure.

In order to improve performance of the surface plasmon resonance sensor using the optical fiber, coupling between a guided mode propagating inside the optical fiber and a plasmon mode of a metal material should be maximized. A plasmonic structure on the optical fiber surface can increase interaction between light and a medium by generating strong electromagnetic fields due to surface plasmon resonance.

The optical fiber for the surface plasmon resonance sensor may have a structure as shown in FIG. 8 to FIG. 11 to generate a strong interaction between a guided mode propagating inside the optical fiber and a plasmon mode.

Referring to FIG. 8, a hetero-core structure is a structure in which two optical fibers having different core sizes are connected. This structure may allow a mode (which goes out of cladding while a light moves from a large core to a small core) to interact with a plasmonic mode. The hetero-core structure has a disadvantage in that sensing sensitivity is low because a change in refractive index is small.

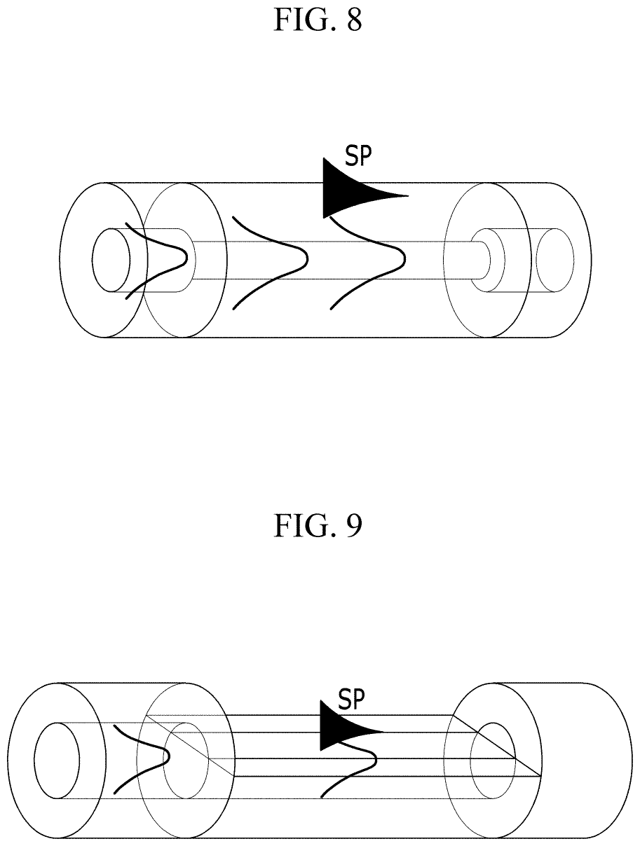

Referring to FIG. 9, a D-shaped SPR probe structure is one having a D-shaped cross-sectional area by side polishing. This structure may allow a mode (which escape from a core to a cladding due to a D-shaped structure while moving into the optical fiber) to interact with a plasmonic mode. The D-type SPR probe is difficult to implement because they are sensitive according to a size and material of the core.

Referring to FIG. 10, a U-shaped SPR probe structure is one in which an optical fiber is bent. This structure may allow a mode (which escape from a core to a cladding while moving into the optical fiber) to interact with a plasmonic mode. The U-shaped SPR probe structure has a problem in that it is difficult to obtain stable performance because a bending loss is sensitively changed by ambient oscillation.

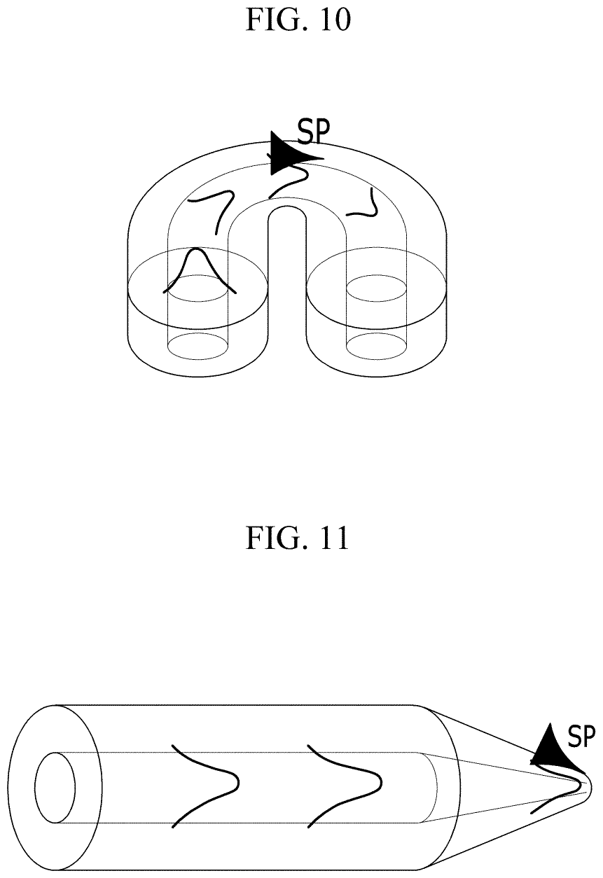

Referring to FIG. 11, a fiber-tip structure is one in which an end of an optical fiber is sharpened and coated with a metal film. The fiber-tip structure is complicated to manufacture because it must be precisely controlled to a micrometer size.

Hereinafter, a surface plasmon resonance sensor using acoustic wave perturbation is described.

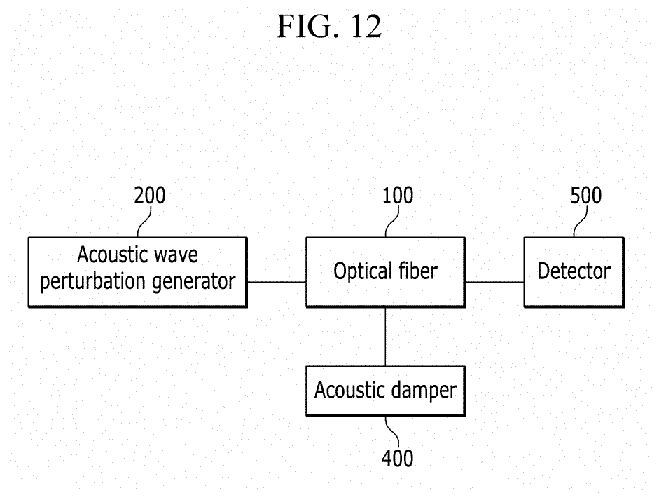

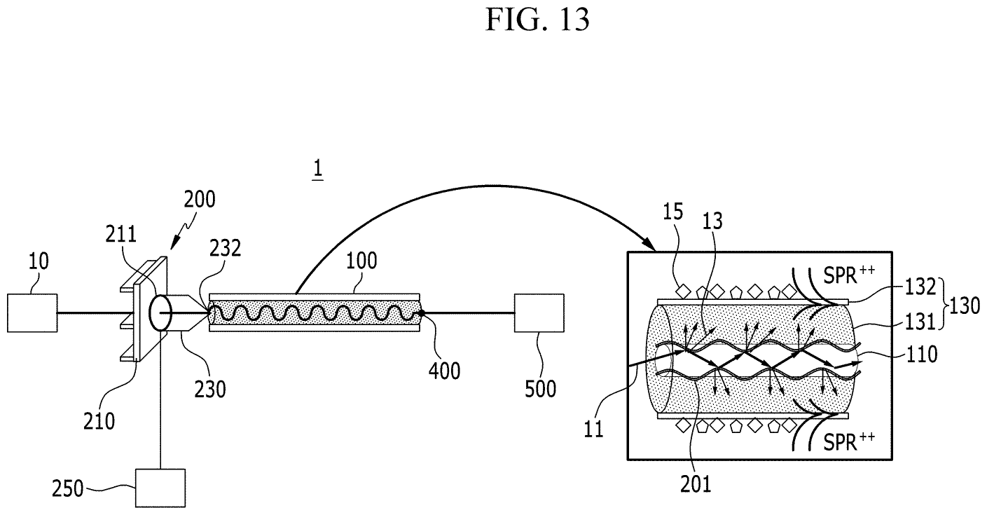

FIG. 12 is a block diagram illustrating a surface plasmon resonance sensor using acoustic wave perturbation according to an exemplary embodiment. FIG. 13 is a schematic diagram illustrating a surface plasmon resonance sensor using acoustic wave perturbation according to an exemplary embodiment.

Referring to FIG. 12 and FIG. 13, a surface plasmon resonance sensor 1 using acoustic wave perturbation according to an exemplary embodiment includes an optical fiber 100, an acoustic wave perturbation generator (or an acoustic transducer) 200, and a detector 500.

The optical fiber 100 may include a core layer 110, and a plasmon resonance layer 130 which is formed to surround an outer surface of the core layer 110.

In an exemplary embodiment, the plasmon resonance layer 130 may be a layer formed of a metallic colloid.

A manufacturing process of the plasmon resonance layer 130 is to remove a cladding layer 131 and a coating layer 132 of the optical fiber 100, and to fill the portion with the metallic colloid. As a result, there is an effect that energy transfer by surface plasmon resonance can be maximized.

In another exemplary embodiment, the plasmon resonance layer 130 may include a cladding layer 131 formed to surround the outer surface of the core layer 110, and a coating layer 132 formed to surround the outer surface of the cladding layer 131.

In an exemplary embodiment, the coating layer 132 may be formed of a metal film.

A manufacturing process of the coating layer 132 is to remove a coating layer of the optical fiber 100, and to coat the portion with the metal film such as gold (Au) or silver (Ag). As a result, there is an effect that energy transfer by surface plasmon resonance can be maximized.

An analyte 15 may be disposed on surface of the plasmon resonance layer 130.

The coating layer 132 may be a layer formed of plasmonic nanostructure. A manufacturing process of the coating layer 132 is to remove a polymer coating layer of the optical fiber 100, and engrave the plasmonic nanostructure having a metal material and a nano-size. As a result, there is an effect that energy transfer by surface plasmon resonance can be maximized.

The acoustic wave perturbation generator 200 may be connected to one side of the optical fiber 100, and may include a metal block 210, an oscillator 230, and a signal generator 250.

In an exemplary embodiment, the metal block 210 may be an aluminum material. The metal block 210 may have a first through hole 211 through which a mode generated by a light source 10 may be passed. The metal block 210 supports the oscillator 230.

In an exemplary embodiment, the oscillator 230 may be a horn-type PZT vibrator. The oscillator 230 may have a second through hole 232 passing through one side and the other side of the oscillator 230, wherein the second through hole 232 of the one side of the oscillator 230 is joined to the first through hole 211, and wherein the second through hole 232 of the other side of the oscillator 230 is joined to the core layer 110 of one side of the optical fiber 100.

In an exemplary embodiment, the signal generator 250 may be a radio frequency (RF) generator. The signal generator 250 transmits a signal to the oscillator 230 coupled to the optical fiber 100 to vibrate the optical fiber 100. As a result, acoustic wave perturbation occurs for a mode propagate within the optical fiber 100. Meanwhile, by controlling the RF of the signal generator 250, a mode 11 which passes through the core layer 110 may pass through the plasmon resonance layer 130 as much as possible.

The detector 500 may be disposed on the other side of the optical fiber 100. The detector 500 may detect the mode 11 that has passed through the inside of the core layer 110.

Referring to FIG. 12 and FIG. 13, the surface plasmon resonance sensor 1 using acoustic wave perturbation according to an exemplary embodiment may further include an acoustic damper 400. In an exemplary embodiment, the acoustic damper 400 may be formed by fixing an epoxy to the other side of the optical fiber 100. Acoustic wave perturbation is removed through the acoustic damper 400.

According to the surface plasmon resonance sensor 1 using the acoustic wave perturbation according to one embodiment, the mode 11 (which passes through the first through hole 211 and the second through hole 232 and which is incident into the core layer 110) is effectively transmitted to the plasmon resonance layer 130 by the acoustic wave perturbation. In addition, since a mode 13 which has passed through the plasmon resonance layer 130 is efficiently coupled to a surface plasmon mode in which an analyte 15 is located, sensitivity for sensing biomolecules (i.e., the analyte 15) is increased.

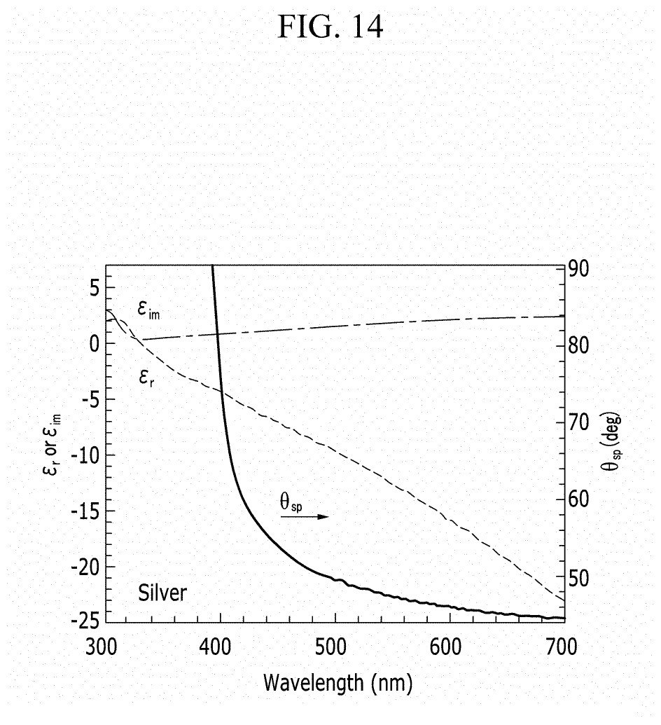

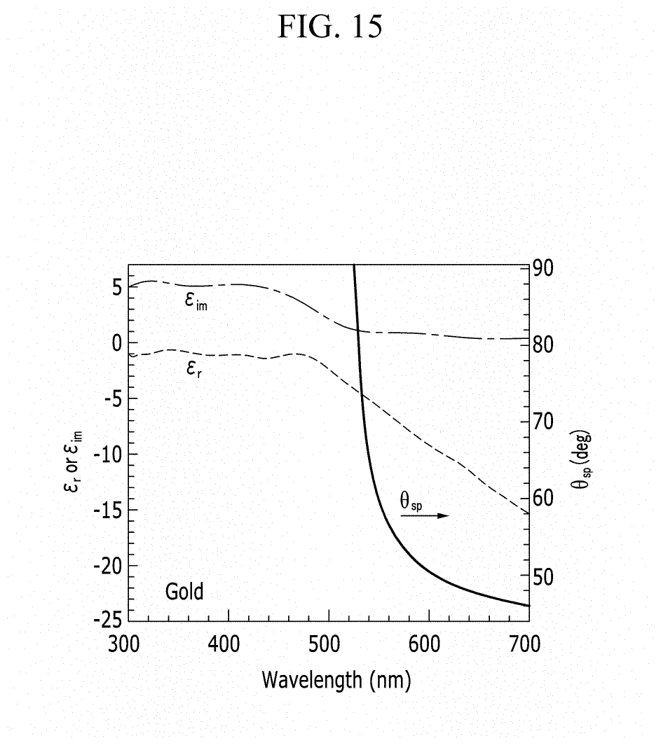

FIG. 14 and FIG. 15 are schematic diagrams illustrating changes in permittivity and angle of surface plasmon resonance according to a metal type.

Referring to FIG. 14 and FIG. 15, since a peak wavelength of a absorption spectrum of a metal changes depending on kinds of the metal, concentration of the colloid, thickness of the film, and the nanostructure, a vibration spectrum of biomolecules must be considered when designing the metal colloid, the metal film, and the nanostructure.

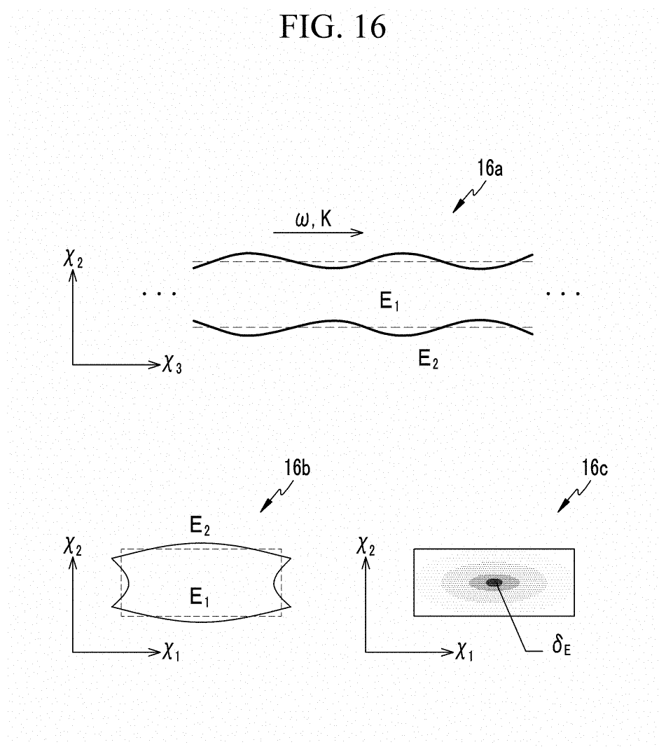

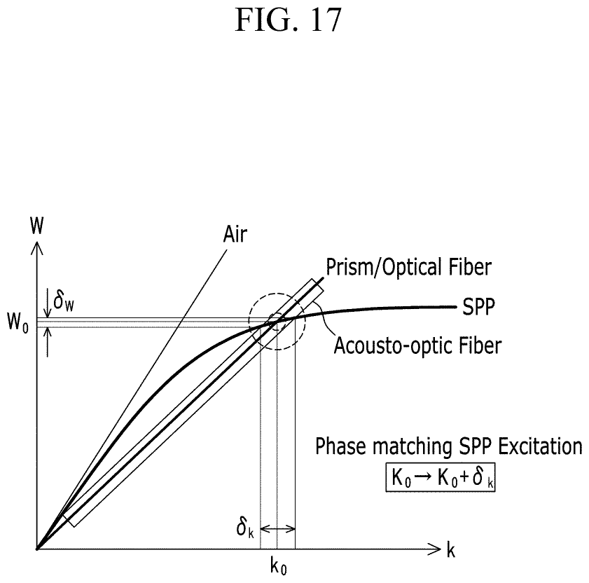

FIG. 16 and FIG. 17 are schematic diagrams illustrating changes in a propagation constant of incident light and phase matching conditions by acoustic wave perturbation of a surface plasmon resonance sensor according to an exemplary embodiment.

Referring to FIG. 16 and FIG. 17, when acoustic wave perturbation by the acoustic wave perturbation generator 200 occurs, a change in the propagation constant of the mode 11 (which is incident light that has moved into the optical fiber 100) occurs. As a result, the phase matching condition between the mode 11 and the plasmon mode widens, and interaction between the mode 11 (which is incident light that has moved into the optical fiber 100) and the plasmon mode is further increased.

More specifically, since acoustic wave perturbation by the acoustic wave perturbation generator 200 causes the mode 11 (which has moved into the optical fiber 100) to move to the plasmon resonance layer 130 of sections with biomolecules, a coupling signal of the mode 11 and the plasmon mode greatly increases.

In addition, acoustic wave perturbation by the acoustic wave perturbation generator 200 changes the propagation constant of the mode 11, and widens the phase matching condition between the mode 11 (which has moved into the optical fiber 100) and the plasmon mode to make surface plasmon resonance stronger and more sensitive.

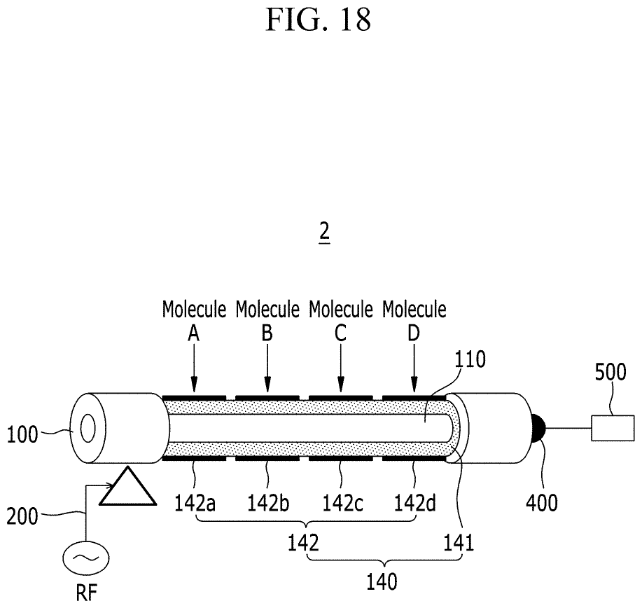

FIG. 18 is a schematic diagram illustrating a multi-channel surface plasmon resonance sensor using acoustic wave perturbation according to an exemplary embodiment.

Referring to FIG. 18, a multi-channel surface plasmon resonance sensor 2 using acoustic wave perturbation according to an exemplary embodiment includes an optical fiber 100, an acoustic wave perturbation generator 200, and a detector 500. The multi-channel surface plasmon resonance sensor 2 has the same configuration except for a plasmon resonance layer 140 of the surface plasmon resonance sensor 1 described above. Therefore, only the plasmon resonance layer 140 (as shown in FIG. 18) will be described below.

The optical fiber 100 may include a core layer 110 and a plasmon resonance layer 140 which is formed to surround an outer surface of the core layer 110. The plasmon resonance layer 140 may include a cladding layer 141 formed to surround the outer surface of the core layer 110, and a plurality of coating layers 142a, 142b, 142c, and 142d spaced apart from each other by a predetermined distance and formed to surround the outer surface of the cladding layer 141.

The spacing of each of the plurality of coating layers 142a, 142b, 142c, and 142d may be variously changed by a designer.

In another exemplary embodiment, the plurality of coating layers 142a, 142b, 142c, and 142d may be layers formed of a metal film such as gold (Au) or silver (Ag). A manufacturing process may be the same as the manufacturing process of the coating layer 132 of the surface plasmon resonance sensor 1.

The plurality of coating layers 142a, 142b, 142c, and 142d may be formed with a plasmonic nanostructure. A manufacturing process may be the same as the manufacturing process of the coating layer 132 of the surface plasmon resonance sensor 1.

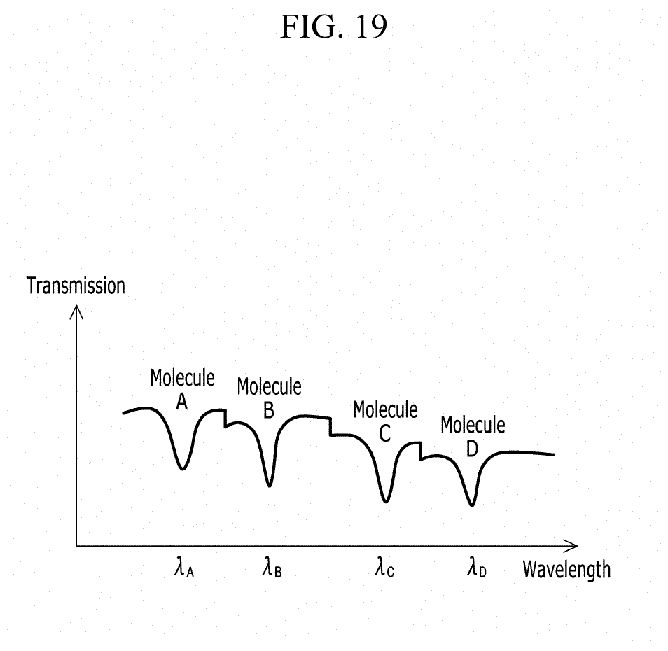

FIG. 19 is a schematic diagram illustrating a spectrum of a multi-channel surface plasmon resonance sensor using acoustic wave perturbation according to an exemplary embodiment.

Referring to FIG. 19, according to the multi-channel surface plasmon resonance sensor 2 using acoustic wave perturbation according to another embodiment, a plurality of analytes are arranged in a line on each of a plurality of coating layers 142a, 142b, 142c, and 142d to detect the plurality of analytes at once. Further, the plurality of analytes have different resonance wavelengths, therefore, it is possible to measure the plurality of analytes on one spectrum at one time.

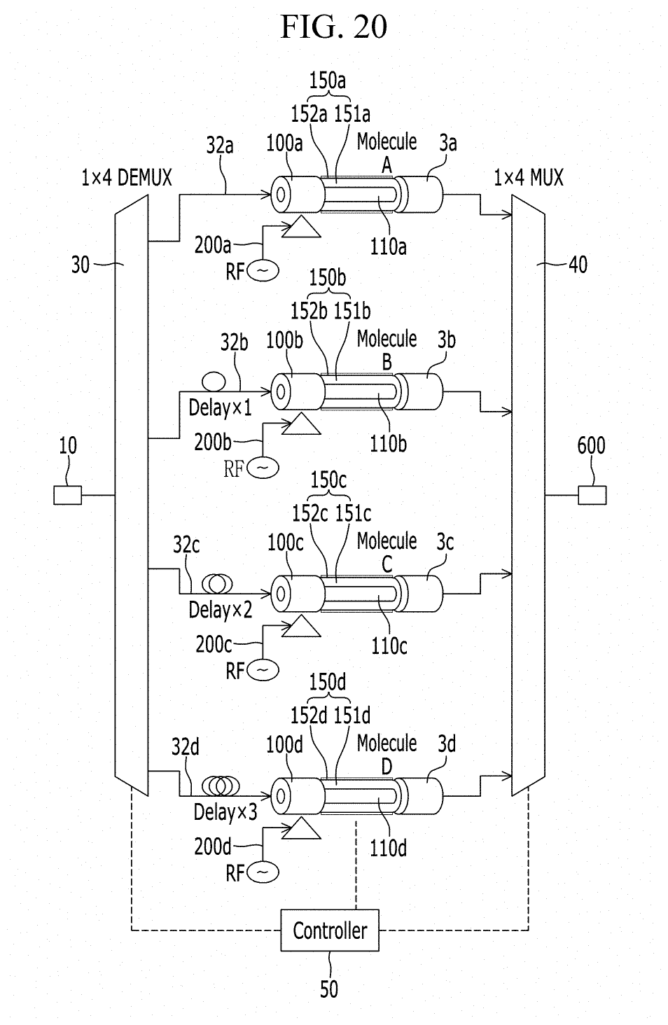

FIG. 20 is a schematic diagram illustrating a multi-channel surface plasmon resonance sensing system using acoustic wave perturbation and time delay according to another exemplary embodiment.

Referring to FIG. 20, a multi-channel surface plasmon resonance sensing system using acoustic wave perturbation and time delay according to another embodiment includes a plurality of surface plasmon resonance sensors 3a, 3b, 3c, and 3d, a demultiplexer 30, a multiplexer 40, a controller 50, and a detector 600.

The plurality of surface plasmon resonance sensors 3a, 3b, 3c, and 3d are sensors for sensing analytes. Each of the plurality of surface plasmon resonance sensors 3a, 3b, 3c, and 3d may include optical fibers 100a, 100b, 100c, and 100d including core layers 110a, 110b, 110c, and 110d, plasmon resonance layers 150a, 150b, 150c, and 150d which are formed to surround an outer surface of the core layers 110a, 110b, 110c, and 110d and on which the analytes are disposed, and acoustic wave perturbation generators 200a, 200b, 200c, and 200d connected to one side of the optical fibers 100a, 100b, 100c, and 100d and generating acoustic wave perturbation to a mode which enters into the core layers 110a, 110b, 110c, and 110d to allow the mode to exit the plasmon resonance layers 150a, 150b, 150c, and 150d.

Each of the plurality of surface plasmon resonance sensors 3a, 3b, 3c, and 3d are the same as the surface plasmon resonance sensor 1 according to another embodiment described above. Therefore, a detailed description of the structure will be omitted.

The demultiplexer 30 may be a 1×N wavelength division multiplex (WDM), and may distribute light from one light source to a plurality of surface plasmon resonance sensors 3a, 3b, 3c, and 3d by instruction of the controller 50.

The demultiplexer 30 and each of the plurality of surface plasmon resonance sensors 3a, 3b, 3c, and 3d may be connected by optical fibers 32a, 32b, 32c, and 32d having different lengths. The time at which light is input to each of the sensors 3a, 3b, 3c, and 3d varies depending on the lengths of the optical fibers 32a, 32b, 32c, and 32d. As a result, it is possible to sense analytes having similar resonance wavelengths with a time difference. The lengths of the optical fibers 32a, 32b, 32c, and 32d may be variously changed by the designer. A delaying time when light is input to the sensors 3a, 3b, 3c, and 3d may be produced by applying a filter in addition to adjusting the lengths of the optical fibers 32a, 32b, 32c, and 32d.

The multiplexer 40 integrates a plurality of signals detected from the plurality of surface plasmon resonance sensors 3a, 3b, 3c, and 3d into one, and transmits them to an output line.

The controller 50 controls the demultiplexer 30 to distribute light from one light source to a plurality of surface plasmon resonance sensors 3a, 3b, 3c, and 3d corresponding to N channels. The controller 50 controls the acoustic wave perturbation generators 200a, 200b, 200c, and 200d so that acoustic wave perturbation occurs in the light distributed to the sensors 3a, 3b, 3c, and 3d from the demultiplexer 30.

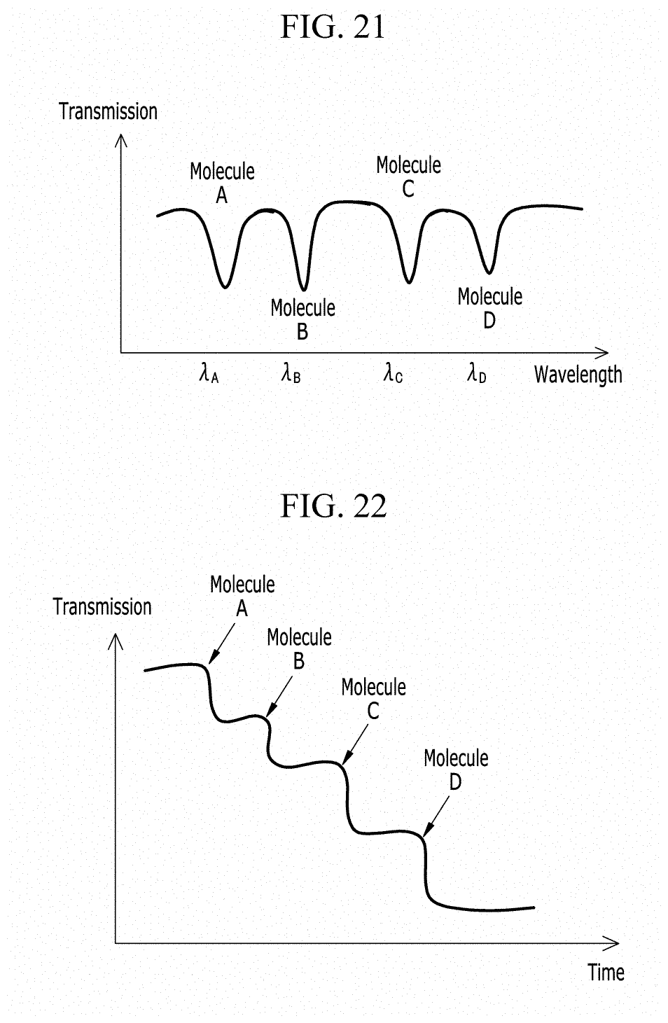

FIG. 21 and FIG. 22 are schematic diagrams illustrating a spectrum of a multi-channel surface plasmon resonance sensing system using acoustic wave perturbation and time delay according to an exemplary embodiment.

According to the multi-channel surface plasmon resonance sensing system using acoustic wave perturbation and time delay according to another embodiment, it is possible to detect molecules having different resonance wavelengths at one time as shown in FIG. 21. As shown in FIG. 22, molecules having similar resonance wavelengths may be measured by separating analytes through time delay.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A sensor for measuring an analyte using surface plasmon resonance, comprising:
an optical fiber including a core layer, and a plasmon resonance layer which is formed to surround an outer surface of the core layer and on which the analyte is disposed;
an acoustic wave perturbation generator connected to one side of the optical fiber, and generating acoustic wave perturbation to a mode which enters into the core layer to allow the mode to exit the plasmon resonance layer; and
a detector for detecting the mode passing through inside of the core layer.

2. The sensor of claim 1, wherein
the plasmon resonance layer is formed of a metallic colloid.

3. The sensor of claim 1, wherein
the plasmon resonance layer includes:
a cladding layer formed to surround the outer surface of the core layer, and
a coating layer formed to surround the outer surface of the cladding layer.

4. The sensor of claim 3, wherein
the coating layer is formed of a metal film.

5. The sensor of claim 3, wherein
the coating layer is formed of a plasmonic nanostructure.

6. The sensor of claim 1, wherein
the acoustic wave perturbation generator includes:
a metal block having a first through hole;
an oscillator having a second through hole passing through one side and the other side of the oscillator, wherein the second through hole of the one side is joined to the first through hole, and wherein the second through hole of the other side is joined to the core layer of one side of the optical fiber; and
a signal generator for transmitting a signal to the oscillator to generate acoustic wave perturbation to a mode which passes through the first through hole and the second through hole and which enters into the core layer.

7. The sensor of claim 8, further comprising
an acoustic damper connected to the other side of the optical fiber to eliminate the acoustic wave perturbation.

8. The sensor of claim 1, wherein
the plasmon resonance layer includes:
a cladding layer formed to surround the outer surface of the core layer; and
a plurality of coating layers spaced apart from each other by a predetermined distance, and formed to surround the outer surface of the cladding layer.

9. The sensor of claim 6, wherein
the plurality of coating layers are formed of a metal film.

10. The sensor of claim 6, wherein
the plurality of coating layers are formed of a plasmonic nanostructure.

11. The sensor of claim 8, wherein
the acoustic wave perturbation generator includes:
a metal block having a first through hole;
an oscillator having a second through hole passing through one side and the other side of the oscillator, wherein the second through hole of the one side is joined to the first through hole, and wherein the second through hole of the other side is joined to the core layer of one side of the optical fiber; and
a signal generator for transmitting a signal to the oscillator to generate acoustic wave perturbation to a mode which passes through the first through hole and the second through hole and which enters into the core layer.

12. The sensor of claim 8, further comprising
an acoustic damper connected to the other side of the optical fiber to eliminate the acoustic wave perturbation.

13. A system for sensing a plurality of analytes using a plurality of surface plasmon resonance sensors, comprising:
a plurality of surface plasmon resonance sensors each including an optical fiber including a core layer, and a plasmon resonance layer which is formed to surround an outer surface of the core layer and on which the analytes are disposed, and an acoustic wave perturbation generator connected to one side of the optical fiber and generating acoustic wave perturbation to a mode which enters into the core layer to allow the mode to exit the plasmon resonance layer;
a demultiplexer for distributing light from a light source to the plurality of surface plasmon resonance sensors; and
a controller for operating the demultiplexer so that the light is distributed to each of the plurality of surface plasmon resonance sensors, and for operating the acoustic wave perturbation generator so that the acoustic wave perturbation occurs in the light distributed to each of the plurality of surface plasmon resonance sensors from the demultiplexer.

14. The system of claim 11, wherein
the demultiplexer and each of the plurality of surface plasmon resonance sensors are connected by optical fibers having different lengths.

15. The system of claim 11, wherein
the plasmon resonance layer is formed of a metal colloid.

16. The system of claim 11, wherein
the plasmon resonance layer includes:
a cladding layer formed to surround the outer surface of the core layer; and
a coating layer formed to surround the outer surface of the cladding layer.

17. The system of claim 14, wherein
the coating layer is formed of a metal film.

18. The system of claim 14, wherein
the coating layer is formed of a plasmonic nanostructure.
